(19) United States
(12) Patent Application Publication
(10) Pub. No.: US 2020/0345299 A1
(43) Pub. Date: Nov. 5, 2020

(54) Monitoring Patient Compliance with a Dosage Regimen

(71) Applicant: Closed Loop Medicine Ltd., Cambridge (GB)

(72) Inventors: Paul Goldsmith, London (GB); Felicity Kate Sartain, London (GB); Richard Edward Knight, London (GB); Hakim Adam Yadi, London (GB); David Cox, London (GB)

(73) Assignee: Closed Loop Medicine Ltd., Cambridge (GB)

(21) Appl. No.: 16/860,659

(22) Filed: Apr. 28, 2020

Related U.S. Application Data
(60) Provisional application No. 62/841,966, filed on May 2, 2019.

(51) Int. Cl. A61B 5/00; G16H 20/10; G16H 10/60; G06Q 30/00; G16H 50/70; G16H 50/20; G16H 10/20; A61B 5/16; A61J 1/03

(52) U.S. Cl. CPC A61B 5/4833; G16H 20/10; G16H 10/60; G06Q 30/0185; G16H 50/70; G06K 19/0723; G16H 10/20; A61B 5/165; A61J 1/035; A61B 5/002; G16H 50/20

(57) ABSTRACT

The present disclosure relates to monitoring patient compliance with a dosage regimen. In some aspects, a patient unique identifier, which enables a patient to be uniquely identified and associated with a patient record, is received. A message is received. The message includes a package unique identifier that enables a package that a drug administered to the patient was stored within to be uniquely identified. The message includes at least one pair of values, wherein each pair of values corresponds to an administration event and indicates an amount of the drug administered to the patient and a time of administration of the amount of the drug to the patient. The drug administered to the patient using the package unique identifier is identified. The identity of the drug and the at least one pair of values are stored against the patient record, which includes a dosage regimen associated with the patient.

### CROSS-REFERENCE TO RELATED APPLICATIONS

This application claims priority to U.S. Provisional Application No. 62/841,966, entitled “Monitoring Patient Compliance with a Dosage Regimen,” filed May 2, 2019, which is hereby incorporated by reference.

### BACKGROUND

Current medical practice involves generating a prescription that sets out a dosage regimen for a patient to follow. The dosage regimen will typically specify a drug, an amount of the drug to be administered per dose and a frequency with which the drug should be administered. In many cases the patient will self-administer the drug, in which case the medical practitioner must rely on the patient correctly reporting their behaviours to assess patient compliance with the prescription.

In practice patients often do not follow a prescription correctly. A patient can forget to administer a drug on a particular occasion, administer an incorrect amount of a drug, or administer the drug at a frequency that is not consistent with the prescription, to name just a few possibilities. A patient can also inaccurately report their adherence to a treatment regimen, as their perception of the degree to which compliance has been achieved may not in fact reflect the level of compliance that was actually achieved in practice.

A typical course of treatment can last several weeks or months, meaning that the patient will be unlikely to be able to recall specific instances of deviation from the prescription if asked. Currently, a medical practitioner thus has to assume that a patient has administered a drug according to the prescription when the patient returns to the medical practitioner to review progress. This means that any future prescription issued by the medical practitioner is based on an assumed administration of the drug, rather than an actual administration of a drug. This is undesirable as it can lead to sub-optimal prescription in second and subsequent courses of treatment.

For example, a patient that has incorrectly followed a prescription by under-administering a drug may present as having a lesser response to the drug than expected, leading to a new prescription that specifies a higher dosage of the drug than the patient would need if they were to follow the prescription correctly.

What is needed is a technique for determining the degree to which a patient has complied with a prescription, which activity may be referred to herein as ‘assessing compliance’ or similar. The technique would advantageously be simple and easy for the patient to use, to avoid it becoming burdensome. The technique would preferably also be objective, and not depend on the patient's subjective assessment of their compliance. The technique would also preferably enable data analysis techniques to be applied to data gathered in relation to a patient population, in order to gain new insights regarding dosage regimens and/or to provide tailored, patient-specific dosage regimens.

### SUMMARY

Systems and methods for monitoring patient compliance with a dosage regimen are described. A patient record containing a unique identifier associated with the patient is provided, where the record is created in a patient record database as part of a patient enrolling or onboarding process. The patient record also includes a dosage regimen associated with the patient.

At the beginning of a course of treatment according to the dosage regimen, a package containing one or more drugs for administration in accordance with the dosage regimen is dispensed to the patient. On each occasion where the patient administers the one or more drugs, a patient data processing device records the administration of the one or more drugs as an ‘administration event’. The patient data processing device transmits a message to another data processing device, e.g. a Cloud-based server, where the message contains details about the administration event including an amount of the drug(s) administered to the patient and a time of administration of the amount of the drug(s) to the patient. This information is stored by the data processing device against the patient record, thereby generating an objective record of patient compliance. This record can be accessed by a healthcare professional, preferably upon provision of appropriate access credentials. The recorded compliance can be used by the data processing device to generate a recommendation for modifications to the dosage regimen. In some scenarios, the data processing device can update the dosage regimen stored in the patient record based on the recommended modification, such that the patient is subsequently dispensed medication according to the modified dosage regimen.

In a first aspect, a computer-implemented method for monitoring the adherence of a patient to a dosage regimen the method comprises: receiving, by a data processing device, a patient unique identifier that enables the patient to be uniquely identified and associated with a patient record; receiving, by the data processing device, a message, wherein the message includes: a package unique identifier that enables a package that a drug administered to the patient was stored within to be uniquely identified; and at least one pair of values, each pair of values corresponding to an administration event and indicating an amount of the drug administered to the patient and a time of administration of the amount of the drug to the patient; the method further comprising: identifying, by the data processing device, the drug administered to the patient using the package unique identifier; and storing, in a database, the identity of the drug and the at least one pair of values against the patient record, the patient record including the dosage regimen associated with the patient.

In a second aspect, a non-transitory computer-readable medium comprises program instructions that, when executed by a data processing device, cause the data processing device to perform the following actions: receive a patient unique identifier that enables the patient to be uniquely identified and associated with a patient record; receive a message, wherein the message includes: a package unique identifier that enables a package that a drug administered to the patient was stored within to be uniquely identified; and at least one pair of values, each pair of values corresponding to an administration event and indicating an amount of the drug administered to the patient and a time of administration of the amount of the drug to the patient; identify the drug administered to the patient using the package unique identifier; and store, in a database, the identity of the drug and the at least one pair of values against the patient record, the patient record including the dosage regimen associated with the patient.

In a third aspect, a data processing device is configured to perform the following actions: receive a patient unique identifier that enables the patient to be uniquely identified and associated with a patient record; receive a message, wherein the message includes: a package unique identifier that enables a package that a drug administered to the patient was stored within to be uniquely identified; and at least one pair of values, each pair of values corresponding to an administration event and indicating an amount of the drug administered to the patient and a time of administration of the amount of the drug to the patient; identify the drug administered to the patient using the package unique identifier; and store, in a database, the identity of the drug and the at least one pair of values against the patient record, the patient record including the dosage regimen associated with the patient.

A package has an associated package unique identifier that uniquely identifies the package. The unique identifier associated with the package enables any given package to be distinguished from all other packages. It is important to appreciate that the package unique identifier is different from a batch identifier, as the latter identifies a group of packages, e.g. as may be manufactured and/or shipped together, whereas the package unique identifier identifies a specific package uniquely. A package may thus have both a package unique identifier and a (different) batch unique identifier.

The package unique identifier can also take many forms, including but not limited to any combination of: a NFC chip embedded in, or affixed to, the package, and/or a visual identifier such as a QR code or a barcode printed on the package, and the like. The package unique identifier is preferably readable by an appropriately equipped data processing device, such as a patient data processing device, and may also be human-readable.

Packages as discussed herein can take many forms, including a carton, a blister pack, a dosette box, a bottle, a tube, a syringe, a pre-filled syringe, a cartridge, a vial or a canister.

A patient data processing device is described. A user uses the patient data processing device, such as a mobile phone, laptop, tablet or other such device, to capture each instance of administration of a drug to a patient. The user may be the patient, or a person assisting with the administration of the drug, etc. The drug is identified using the package unique identifier discussed above. This information is transmitted to a data processing device that has access to a patient record as discussed below, where it is stored against the patient record.

The package unique identifier can be entered manually into the patient data processing device, e.g. via an appropriately configured software application installed on the data processing device having a user interface of the type known in the art per se, or via a web form rendered by a web browser application. Alternatively, the patient data processing device can be configured to receive the package unique identifier in an automatic manner, e.g. reading a NFC chip embedded in the package using a NFC reader of the patient data processing device, or scanning a QR code or barcode using a camera of the patient data processing device. Other mechanisms for receiving the package unique identifier will be apparent to the skilled person having the benefit of the present disclosure. Reference is also made here to FIG. 6 and its accompanying description.

Each patient is provided with a patient unique identifier. This is any identifier that enables a particular patient to be uniquely identified. The unique identifier can be human-readable and machine-readable, or it can be machine-readable only. The unique identifier can be assigned to the patient as part of an on-boarding or enrolling process, which process involves the gathering of patient details such as name, address, etc. and generating a unique identifier for the patient. The unique identifier may be assigned to the patient by an external authority such as a governmental organisation. An example of an external authority in the context of the United Kingdom healthcare system is the National Health Service, NHS, where the patient unique identifier is a NHS number. Suitable patient unique identifiers will be apparent to the skilled person having the benefit of the present disclosure. The unique identifier may be assigned to the patient as part of the enrolment process onto a clinical trial.

Each patient also has an associated patient record. The patient record is an electronic record that contains information about the patient, the information including at least the unique identifier. Any information pertinent to the patient can also be stored in the patient record, including but not limited to any combination of: a patient name, a patient address, a patient age, a medical history of the patient, a name and/or address of a medical practitioner and/or medical institution responsible for providing care for the patient, etc.

The patient record can be stored in a patient record database that is accessible to a data processing device, e.g. a Cloud-based server. The patient unique identifier can be used in a query of the patient record database to retrieve the patient record and/or any of the information contained therein.

The data processing device is communicatively coupled to the patient data processing device via one or more communication channels. The communication channel(s) can take the form of any suitable mechanism for enabling communication between computational devices, including but not limited to data transmission over a public network such as the internet, transmission over a cellular network, or a combination thereof. Other suitable communication channels will be readily identified by the skilled person having the benefit of the present disclosure.

The patient data processing device is configured to transmit a patient unique identifier of the type discussed above to the data processing device over the one or more communication channels. The patient data processing device may obtain the patient unique identifier from the patient during the aforementioned enrolment or on-boarding process, in which the patient may be required to register with a compliance monitoring platform to create a patient account, and subsequently log in to the patient account using authentication credentials such as a password and/or biometric identifier.

Upon successful authentication, the patient unique identifier may be made available to the patient data processing device, e.g. by retrieving it from a remote location and caching it in a local memory store of the patient data processing device, or by storing data inputted directly into the patient data processing device by the patient, e.g. in a field labelled ‘patient identifier’, ‘username’, or similar. Many variants of such techniques for enrolling a person with a service and subsequently identifying that person on log in to the service are known in the art per se and consequently are not described in detail here. Any such variant can be used.

The patient data processing device is also configured to transmit a message to the data processing device upon detection of an administration event. As used herein, an administration event refers to the administration of a drug to the patient.

It will be appreciated that some treatment regiments require a patient to administer multiple distinct drugs. In such cases, administration of each drug can be treated as a separate administration event, such that one message is generated in respect of each drug administered. Alternatively, the administration of the multiple drugs can be treated as a single administration event. It may be preferred to treat administration of each drug as a separate administration event in situations where each drug is administered in a separate form, e.g. capsules containing a first drug and separate capsules containing a second drug. This enables compliance with each individual drug to be monitored. It may be preferred to treat administration of multiple drugs as a single administration event in situations where multiple drugs are administered in a mixed form, such as a liquid solution containing two or more drugs. This is because it is not possible for the patient to separately administer just one of the constituent drugs in this scenario, such that there is little value in attempting to track administration of each drug separately.

The administration of more than one discrete item containing the drug, e.g. the taking of two or more capsules containing the same drug, can be treated as a single administration event due to the fact that the patient has only administered a single drug during this event.

The patient data processing device is configured to receive the package unique identifier in the manner discussed above and to transmit the package unique identifier to the data processing device over the one or more communication channels. The package unique identifier is transmitted as part of the message that is generated on detection of an administration event.

The message also includes a pair of values that correspond to an administration event. The pair of values include an amount of the drug administered to the patient during the administration event and a time at which that amount of drug was administered to the patient.

The message is structured such that a pair of values corresponds to a particular administration event. For example, the message may include the following fields:
1) a ‘type’ field indicating that the message is an administration event message;
2) an ‘amount’ field specifying an amount of drug administered in a suitable unit (e.g. milligrams, mg), or including data from which an amount of drug administered can be determined;
3) a ‘time’ field specifying the time at which the drug was administered (e.g. in a UTC format), which can include both a date and time; and
4) a ‘package ID’ field including the package unique identifier.

It will be appreciated that this list is exemplary and non-exhaustive, such that other fields may be present in the message. The names for each field given above are also purely exemplary and deviation is therefore possible. Here, a ‘field’ should be understood as any machine-readable mechanism for storing data in a structured manner. An exemplary implementation of the message is thus a transmission containing an XML format file having a structure that supports provision of information as required by items 1) to 4) above. Variations will be apparent to a skilled person having the benefit of the present disclosure.

In addition to the above, the patient data processing device may be configured to gather any combination of the following data and to transmit this data to the data processing device:
A) One or more environmental parameters that characterise the environment local to the patient at the time of administering a drug. The environmental parameters include, but are not limited to, any one of more of: an ambient light level, e.g. as measured by a camera of the patient data processing device; a location, e.g. as measured by a GPS sensor of the patient data processing device; a temperature, e.g. as measured by a thermometer of the patient data processing device; a pressure, e.g. as measured by a barometer of the patient data processing device; an ambient sound level, e.g. as measured by a microphone of the patient data processing device; an ambient magnetic field strength, e.g. as measured by a magnetometer of the patient data processing device; and/or an ambient moisture level, e.g. as measured by a moisture sensor of the patient data processing device. The environmental parameters may be experienced by the patient at the time of administering the drug, and/or they may be historical environmental parameters experienced by the patient over a time period preceding the administration event.
B) Behavioural data indicative of a behaviour of the patient. The behavioural data can include, but is not limited to, any one or more of: data indicative of patient exercise levels, e.g. accelerometer data generated by an accelerometer of the patient data processing device; sleep quality data of the type known in the art per se; direct patient feedback such as results from a questionnaire completed by the patient, perhaps using the patient data processing device; patient reported pain levels, e.g. as manually input to the patient data processing device; and patient reported stress levels, e.g. as manually input to the patient data processing device.
C) Physiological data relating to the patient. The physiological data can include, but is not limited to, any one of more of: a blood pressure; a biomarker level; a blood sugar level; a respiratory rate; a heart rate; a skin moisture level; a skin pH value; and a body temperature. A person skilled in the art will readily identify alternative physiological parameters that can be measured and provided to the data processing device. In some cases the physiological data may be measured directly by the patient data processing device, and in others the patient data processing device may be in communication with a separate physiological parameter measuring device that transmits the physiological data to the patient data processing device.
D) Historic package data relating to the history of the package. This can include one or more of the past location of the package, the current location of the package and/or the physical condition of the package. Historic package data may be provided by an accelerometer embedded in the package, which accelerometer gathers data indicative of the acceleration that the package has undergone. Package data of this type is gathered based on the insight that the efficacy of some drugs, particularly those relating to biologic therapies, are thought to depend on the degree to which the drug has been agitated or otherwise moved before administration. In some cases excess movement, particularly violent movement, may damage proteins critical to the activity of the biologic, rendering it less effective. In other cases, agitating or otherwise moving the drug prior to administration may be beneficial, e.g. in the case of a suspension where a homogeneous mixture is preferred directly before administration. Data relating to the historical motion of the package is thus expected to be of use in monitoring the efficacy of a drug. The historic and/or current location of the package can be gathered by a location sensor embedded in the package, e.g. a GPS sensor. The location of the package can be correlated with other pertinent location information in order to gain insights into the history of the package. For example, examination of the package location history may reveal that the package has been in a controlled environment, e.g. a prison. As another example, examination of the package location history may reveal that the package was located remotely from a patient data processing device at the time at which a drug was removed from the package. In this way, location history may be used to gain additional understanding of the administration of drugs to patients.

It will be appreciated that in some scenarios it is necessary to notify the data processing device about more than one administration event. For example, the patient data processing device may be reliant upon a cellular network or WiFi network to transmit data to the data processing device, and may have been unable to obtain connectivity to such networks for a period of time during which more than one administration event occurred. In such cases, the data processing device may transmit a message as described above containing a plurality of pairs of values of the type discussed above, with each pair of values corresponding to a particular administration event.

It is preferred that an encrypted channel is used where any information confidential to the patient is being transmitted and/or where control commands are received by the patient data processing device. Techniques for establishing encrypted channels over a public network, e.g. a SSH channel, are known per se and hence are not discussed in detail here.

The message is transmitted by the patient data processing device to the data processing device over the one or more channels. The data processing device is configured to receive the message and to use the package unique identifier contained therein to identify the drug administered to the patient. The data processing device may, for example, query a database using the package unique identifier, with the result of the query including an identification of the drug contained within the package corresponding to the package unique identifier.

The data processing device is configured to store the identity of the drug and the at least one pair of values against the patient record. It will be appreciated that a patient record therefore contains one or more data points, each data point comprising a drug amount and corresponding administration time.

The patient record can be used by a healthcare professional and/or the data processing device to assess compliance with the dosage regimen. A compliance score may be generated, indicating the degree to which the patient has complied with the dosage regimen. The compliance score may be stored by the data processing device against the patient record.

The data processing device can also be configured to provide a recommended modification to the dosage regimen based on at least one of: the identity of the drug; the last one pair of values stored in the patient record; the compliance score; and/or any of the additional data discussed above under items A) to D).

The recommendation can include: a recommended increase in a dosage of a drug; a recommended decrease in a dosage of a drug, where a decrease may include stopping administration of the drug entirely; a recommendation to adapt the dosage regimen to incorporate a second drug; a recommendation to adapt the dosage regimen to incorporate a non-drug therapy, e.g. cognitive behavioural therapy, exercise, dietary changes, etc., and/or a recommendation to replace a drug currently prescribed as part of the dosage regimen with a second, different drug. This list is not exhaustive and other suitable recommendations will become apparent to a skilled person having the benefit of the present disclosure.

The data processing device may include, or otherwise make use of, a machine learning component that uses one or more machine learning algorithms to generate the recommendation. The one or more machine learning algorithms may be trained, at least in part, using a plurality of patient records of the type discussed herein.

The data processing device may be configured to generate a modified dosage regimen based on the recommended modification. The data processing device may be further configured to store the modified dosage regimen against the patient record. Preferably, the next time the patient requests a repeat of their prescription, the patient record is queried and the prescription is provided based on the modified dosage regimen. The treatment of the patient is thus adjusted over time based on data relating to actual drug administration events, as opposed to expected or recalled administration events. Improvements in the adaptation of treatment regimens may therefore be expected.

The data processing device can be configured to transmit a message to a second data processing device, the message including the modified dosage regimen. This may facilitate the next prescription being based upon the modified dosage regimen. The second data processing device can be the patient data processing device or a healthcare provider data processing device, e.g. a computer located at a pharmacy, hospital or other such healthcare institution.

The second data processing device can be any one of: a mobile telephone, a tablet computer, a desktop computer, a voice-activated computing system, a laptop, a gaming system, a vehicular computing system, a wearable device, a smart watch, a smart television, and an internet of things device, a medicament-dispensing device and a device including a drug pump.

The second data processing device may be a device that is in direct control of dispensing the drug to the patient, e.g. a medicament dispensing device or a drug pump, such that the modified dosage regimen is applied directly without action being required on the part of either a healthcare professional or the patient.

The amount of the drug administered to the patient can be manually input into the patient data processing device by the patient. Alternatively, the patient data processing device can be configured to obtain the amount of the drug automatically. At least the following techniques are suitable for making this automatic determination.

In a first technique, the patient data processing device is configured to scan the package from which the drug is being dispensed to identify a change in the package indicating an administration event. The patient data processing device can use this change in the package to calculate the amount of the drug that has been administered by the patient.

The scan can include an optical scan where the patient data processing device takes one or more images of the package using an imaging component such as a camera. The patient data processing device can be configured to compare the one or more images to a previous image of the package. The optical scan is carried out after the administration event.

The previous image can be an image of the package captured by the patient data processing device before the administration event, or the previous image can be a stock image of a package identical to the package that the patient has in their possession. In the latter case, the stock image may be retrieved by the patient data processing device based on the package unique identifier, e.g. querying a database of stock images using the package unique identifier and receiving the stock image, or a pointer to the stock image, as a response.

The comparison can include application of one or more image processing algorithms that are capable of identifying a change between the previous image of the package and the one or more images. For example, in the case where the package is a blister pack, the one or more image processing algorithms may be capable of identifying one or more blisters of the blister pack that have been opened during the administration event. Image processing techniques incorporating any form of machine learning may be used. Identification of the one or more opened blisters enables the patient data processing device to identify the capsules(s) that have been administered. For a given package, the amount of a drug within each capsule is known a priori, and this information can be retrieved by the patient data processing device, e.g. based on the package unique identifier, to enable the patient data processing device to calculate an amount of the drug administered during the administration event.

The techniques described in the immediately preceding paragraph can be applied to other package types having multiple discrete sub-units that are opened in order to gain access to a capsule, tablet, etc., such as a dosette box or a carton.

In order to obtain a good quality image of the package, the user of the patient data processing device (typically, but not exclusively, the patient) may be directed by an on-screen utility whilst taking the image(s). The on-screen utility may comprise a frame or other such locating mechanism that the user is directed to align with an edge of the package such that the full extent of the package is captured in the image. Here, a ‘good quality’ image is understood to mean an image that has a good prospect of being processed successfully by the one or more algorithms such that a drug amount can be determined to at least a good confidence level.

In some circumstances the patient data processing device may have limited local processing resources. In these and other cases the patient data processing device can be configured to transmit the one or more images in the message. In such a scenario, the data processing device can be configured to process the one or more images according to one or more algorithms as discussed above in order to calculate an amount of the drug administered to the patient. In these circumstances, transmission of the one or more images to the data processing device is understood to be an example of providing a value that is indicative of an amount of the drug administered to the patient.

In a second technique, the data processing device is communicatively coupled to a weighing device that is configured to calculate at least a change in weight of the package. The patient data processing device may direct the patient to weigh the package before and after the administration event, and receive from the weighing device a first weight value corresponding to the weight of the package before the administration event and a second weight value corresponding to the weight of the package after the administration event. From this, a change in weight can be calculated.

This technique is particular suitable for drugs that are dispensed in packages such as a bottle, a tube, a vial or a canister. The change in weight can be converted into an amount of drug administered by the patient data processing device using an appropriate formula as will be determinable by a skilled person without difficulty. Information such as the density of a solution containing the drug, the concentration of the solution, etc. can be retrieved if needed in this calculation by querying a database using the package unique identifier.

The patient data processing device can be configured to transmit the change in weight to the data processing device, such that the data processing device subsequently determines the amount of drug administered using the change in weight. In this case, the change in weight is understood to constitute a value indicating an amount of the drug administered to the patient.

In a third technique, the package itself may constitute a known quantity of the drug. Examples of such packages include a syringe, a pre-filled syringe and a cartridge. In these cases the amount of drug administered can be determined directly using the package unique identifier, since the amount of the drug contained by the package is known a priori and the entire package contents are administered to the patient in one administration event.

In this case the package unique identifier may be transmitted by the patient data processing device as the value corresponding to the amount of drug administered, with the data processing device being configured to use the package unique identifier to look up the amount of a drug contained by the package in a database. Alternatively, the patient data processing device may be configured to perform this lookup and transmit the result to the data processing device as the value indicating an amount of the drug administered to the patient.

It will be appreciated that the first to third techniques described above are all suitable for use in respect of an unmodified package, in the sense that a package that is manufactured according to known methods can be used with these techniques.

In a fourth technique, which is currently preferred, the package itself is a ‘smart package’ that is provided with mechanism for detecting the extraction of a drug during an administration event. Reference is made here to FIG. 6. The mechanism can take the form of one or more electrical wires that are physically arranged across a surface or surfaces of the package such that retrieval of a drug from the package causes a change to an electrical signal passing through the one or more electrical wires.

The change in signal can be detected by either an integrated package data processing device or the patient data processing device, enabling either device to positively identify that a drug has been extracted from the package. The term ‘change in signal’ is understood to encompass a drop in the signal to a negligible or zero level, as would occur in a break in the at least one electrical wire such that current can no longer flow along it, as well as a reduction in the magnitude of the signal as would be caused by e.g. some of a plurality of wires being broken.

In the case where the package contains more than one compartment each housing a dosage of a drug, e.g. a blister pack containing a plurality of individual capsules or tablets, the one or more electrical wires are arranged across the surface or surfaces of the package such that the package data processing device or patient data processing device is able to determine which of the compartments has been opened. This enables the package data processing device or patient data processing device to identify the tablet(s) or capsule(s) that have been administered during an administration event, such that an amount of drug administered can be directly determined.

The package also comprises a package data processing device that is embedded with the package and coupled to the one or more electrical wires. The package data processing device is configured to provide at least one of the pair of values, and particularly at least the amount of the drug administered to the patient. The package may also comprise a timing circuit that is capable of keeping track of time such that time at which the drug was extracted from the package can be recorded. The time of extraction of the drug from the package may be taken as the time of administration of the drug, as typically the time between extraction and administration is negligible (e.g. seconds or tens of seconds).

The package data processing device is configured to communicate with at least the patient data processing device to transmit this information to the patient data processing device.

The package data processing device can be, for example, a microprocessor coupled to an antenna such as a NFC antenna, Bluetooth antenna, preferably a Bluetooth Low Energy antenna and/or a radio frequency antenna for communicating over a cellular network.

The package may contain an embedded memory unit that is coupled to the microprocessor and configured to store one or more data items, including an identification of a compartment that was opened and the time at which the compartment was opened. The package may contain one or more embedded accelerometers that are configured to gather motion data, which may be stored in the memory unit. This motion data can be used to establish a motion history of the package, as discussed above in respect of item D).

The patient data processing device may be configured to provide an ‘augmented reality’ capability to assist the patient in administering a drug. The augmented reality capability may comprise an overlay on a display of the patient data processing device, which overlay indicates in some manner the specific drug or drugs that the patient should administer in a particular administration event.

For example, in the case of a blister pack, the overlay may comprise a visual element, e.g. a box, frame, arrow, etc., that is displayed on the display of the patient data processing device, where the visual element is located such that it identifies one or more individual blisters that the patient should open in this particular administration event. The patient data processing device may be configured to display assistive text in addition to, or in the alternative of, the visual element, where the assistive text instructs the patient in relation to the current administrative event. Audible instructions corresponding to the assistive text may be provided in addition to, or in the alternative of, the assistive text itself. Exemplary assistive text is as follows: ‘Please take one tablet from a red compartment and one tablet from a blue compartment’. A link, e.g. a hyperlink, may be displayed on a display of the patient data processing device, directing the patient to a resource, e.g. a webpage, containing further information should they require it.

The assistive text, audible instructions and/or visual element may be generated by the patient data processing device based on the dosage regimen currently in effect for the patient. This may be retrieved from the patient record by the following process: transmitting, by the patient data processing device, a request for the current dosage regimen to the data processing device, the request including a patient unique identifier; receiving, by the patient data processing device, a response from the data processing device, the response including the current dosage regimen; generating one or more of a visual element, an assistive text label and audible instructions based on the received current dosage regimen; and any combination of: displaying the visual element on a display of the patient data processing device in a manner that indicates one or more discrete drugs for administration to the patient; displaying the assistive text label on the display of the patient data processing device and/or playing the audible instructions using a speaker of the patient data processing device. Text to speech techniques that are suitable for generating the audible instructions are known per se in the art.

The data processing device can handle requests for a current dosage regimen according to the following process: receiving, by the data processing device, a request for a current dosage regimen from a patient data processing device, the request including a patient unique identifier; identifying, in a database, a patient record corresponding to the patient unique identifier; retrieving, from the patient record, a current dosage regimen; and transmitting, by the data processing device, the current dosage regimen to the patient data processing device.

### BRIEF DESCRIPTION OF THE DRAWINGS

FIG. 1 shows a system suitable for implementing any of the methods described in this specification, in accordance with an embodiment.

FIG. 2 shows a method for storing data relating to an administration event that can be performed by one or more components of the system of FIG. 1, in accordance with an embodiment.

FIG. 3 shows an exemplary patient record in accordance with an embodiment.

FIG. 4 shows a method for providing a recommended modification of a dosage regimen that can be performed by one or more components of the system of FIG. 1, in accordance with an embodiment.

FIG. 5 shows a method for dispensing a package including medication in accordance with a dosage regimen that can be performed by one or more components of the system of FIG. 1, in accordance with an embodiment.

FIG. 6 shows an exemplary drug-containing package in accordance with an embodiment.

### DETAILED DESCRIPTION

The following definitions are provided to assist with the understanding of this specification.

The term “comprises”, and variations thereof, do not have a limiting meaning where these terms appear in the description and claims. Such terms will be understood to imply the inclusion of a stated step or element or group of steps or elements but not the exclusion of any other step or element or group of steps or elements.

The term “consisting of” means including, and limited to, whatever follows the phrase “consisting of”. Thus, the phrase “consisting of” indicates that the listed elements are required or mandatory, and that no other elements may be present for that particular feature.

The term ‘the Cloud’, or equivalently ‘Cloud-based’, should be understood to be a reference to one or more configurable computing resources that can be called upon to perform tasks according to need. The computing resources are located remotely from a user or a data processing device associated with the user and are accessible over a network such as the internet and/or a cellular network.

The term ‘machine-readable’ or ‘machine intelligible’ is understood to refer to data that is stored in a format that is processable by a data processing device. Processing includes but is not limited to one or more of: identifying and displaying one or more data items stored in a machine-readable data structure on a display device; and extracting one or more data items stored in a machine-readable data structure and performing one or more calculations on said data items.

As used herein, the term ‘time’ should be understood as encompassing data entries that identify a date in addition to a measure of hours, seconds and minutes. Thus, 1 Jan. 2019, 08:43:22 (day, month, year, hrs:mins:secs) is understood to fall within the definition of ‘time’ as used herein. An example of a suitable machine-readable format for encoding this information is Coordinated Universal Time, UTC, as known in the art per se.

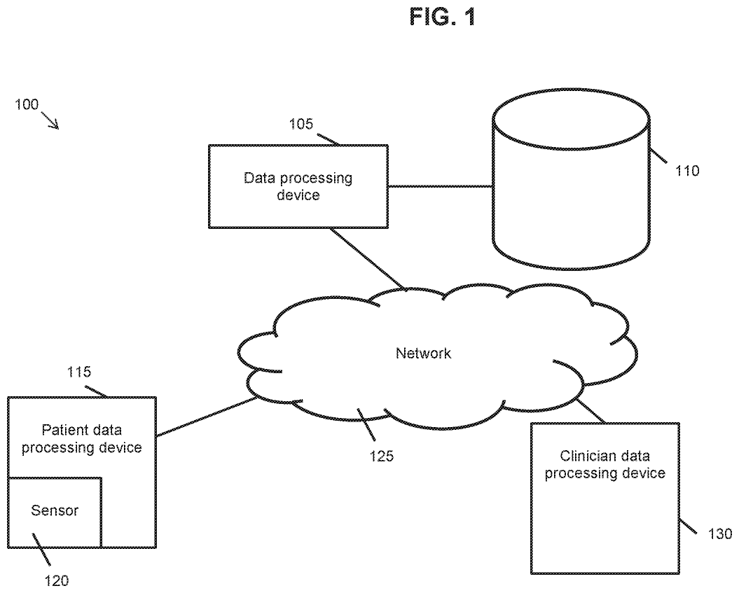

A system 100 suitable for carrying out any of the computer-implemented methods according to the present disclosure is shown in FIG. 1. System 100 includes a data processing device 105 that is communicatively coupled to a database 110 that stores a patient record as discussed earlier in this specification. Database 110 is stored on a storage medium, e.g. a Cloud-based storage medium.

Data processing device 105 comprises at least one processor and is configured to carry out any of the methods described in this specification, or one or more steps thereof. Data processing device 105 is configured to operate in accordance with one or more programmatic instructions stored on a non-transitory computer readable storage medium. Data processing device 105 may be configured to execute one or more machine learning tasks. The machine learning tasks include any combination of: training a model using data from patient records stored in database 110 and/or using a trained model to classify an input such as data from a patient record stored in database 110.

Data processing device 105 can be configured to perform other tasks including: receiving data from a patient data processing device 115; generating a patient record for storage in database 110; amending or otherwise updating a patient record stored in database 110; transmitting information and/or commands to patient device 115 and/or clinician data processing device 130, and the like. Data processing device 105 may include a Cloud-based web server that hosts a website or portal which is accessible to one or both of patient device 115 and clinician data processing device 130.

System 100 also comprises patient data processing device 115. In the present embodiment, patient data processing device 115 is a smartphone, optionally comprising a sensor 120. However, the present disclosure is not limited in this respect and patient data processing device 115 can take many other forms, including but not limited to a mobile telephone, a tablet computer, a desktop computer, a voice-activated computing system, a laptop, a gaming system, a vehicular computing system, a wearable device, a smart watch, a smart television, an internet of things device, a medicament-dispensing device and a device including a drug pump.

Patient data processing device 115 is communicatively coupled to data processing device 105 via a network 125. In the illustrated embodiment network 125 is the internet, but the present disclosure is not limited in this respect and network 125 could be any network that enables communication between patient device 115 and data processing device 105, such as a cellular network, or a combination of the internet and a cellular network.

Patient data processing device 115 is configured to gather data relating to a patient and/or the immediate environment of the patient as discussed herein and to transmit at least some of said gathered data to data processing device 105 in the form of a message of the type discussed herein.

Patient data processing device 115 may gather data using sensor 120, which can be any combination of: a light sensor such as a camera, a temperature sensor, an acoustic sensor such as a microphone, an accelerometer, an air pressure sensor, an airborne particulate sensor, a global positioning sensor, a humidity sensor, an electric field sensor, a magnetic field sensor, a moisture sensor, an air quality sensor and a Geiger counter, and/or any other such sensor capable of determining a characteristic of the patient and/or the patient's immediate environment.

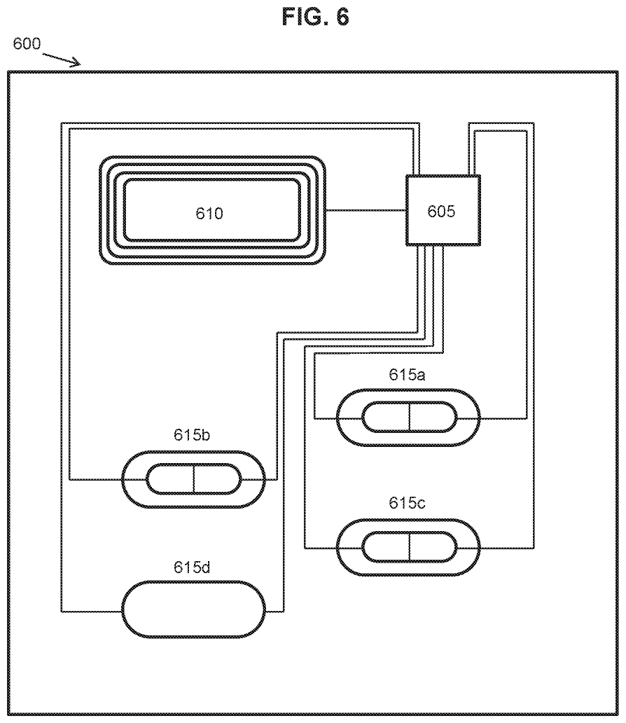

Alternatively, sensor 120 can be omitted from patient data processing device 115. In that case, information about the patient and/or the immediate environment of the patient can be obtained via other mechanisms including manual data entry using a human interface device of patient data processing device 115 and reception of such data from a ‘smart’ package of the type disclosed herein in connection with FIG. 6.

It will be appreciated that system 100 may include more than one patient data processing device that is similar to patient data processing device 115. It is contemplated that a single patient may use more than one patient data processing device to collect data and feed it into system 100.

Patient data processing device 115 may have one or more applications installed on a storage medium associated with the patient data processing device (not shown), the one or more applications configured to control data acquisition via sensor 120 and/or to assist the patient in providing data relating to drug administration events and/or their immediate environment.

System 100 optionally includes a clinician data processing device 130 that is communicatively coupled via network 125 to data processing device 105. The clinician data processing device 130 is broadly similar to patient data processing device 115, offering a similar set of functionality. Specifically, the clinician data processing device 130 enables data relating to administration events and/or the environment experienced by the patient to be collated and transmitted to data processing device 105. Clinician data processing device 130 is contemplated as being physically located at a clinician's premises during its use, such as a doctor's surgery, a pharmacy or any other healthcare institution, e.g. a hospital. Clinician data processing device 130 may include one or more sensors like sensor 120, and/or be configured to control one or more separate sensors like sensor 120, which sensors are capable of gathering information about administration events and/or the environment experienced by the patient.

It is also contemplated that clinician data processing device 130 is typically used by a medically trained person with appropriate data security clearance, such that more advanced functionality may be available than via the patient device 115. For example, the clinician data processing device 130 may be able to access a medical history of the patient, generate a prescription for the patient, amend a patient dosage regimen, place an order for medication, etc. Access to functionality may be controlled by a security policy implemented by data processing device 105.

It is contemplated that system 100 could omit patient data processing device 115 altogether, in which case all reporting of data to data processing device 105 is handled by clinician data processing device 130. This configuration may find particularly utility in situations where a patient is incapable of providing data to data processing device 105 via a patient data processing device 115, e.g. a situation where a patient is incapacitated due to a medical condition. In such embodiments, all functions described herein in connection with patient data processing device 115 would be instead carried out by the clinician data processing device 130.

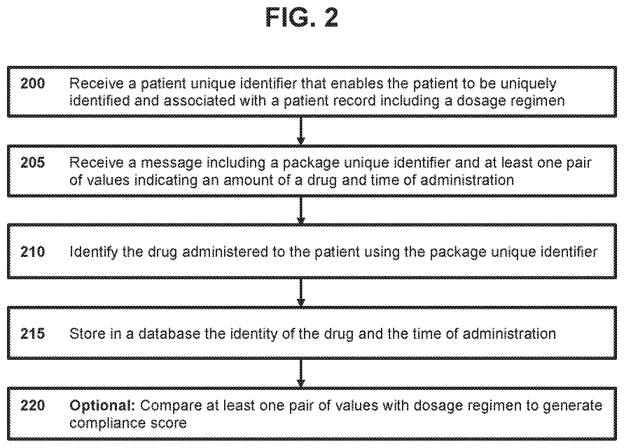

FIG. 2 shows a method suitable for appending a new entry to a patient record that can be performed by data processing device 105 in accordance with an embodiment.

In step 200, data processing device 105 receives a patient unique identifier that enables the patient to be uniquely identified and associated with a patient record including a dosage regimen. The patient unique identifier takes the form as discussed earlier and may be received from the patient data processing device 115 over network 125. Where data is gathered by the data processing device 115, e.g. in accordance with any one or more of items A) to D) above, this data may be gathered by sensor 120 that takes the form of any one or more of the sensors discussed in items A) to D).

In the case where no patient record can be identified using the patient unique identifier, data processing device 105 can be configured to transmit instructions to patient data processing device 115 that cause the patient to undergo an onboarding or enrolment process that results in the creation of a patient record for said patient. After this process, data processing device 105 can repeat step 200 and identify the newly created record.

In step 205, data processing device 105 receive a message including a package unique identifier and at least one pair of values indicating an amount of a drug administered to the patient and a time of administration of the drug. The message and values take the form as discussed above and may be received from the patient data processing device 115.

In the case where the value indicating the amount of the drug administered to the patient is provided in a format that requires additional processing such as the image analysis discussed above in respect of the first technique, data processing device 105 can be configured to perform such additional processing, with the end result that data processing device 105 calculates an amount of the drug administered to the patient.

In step 210, data processing device 105 identifies the drug administered to the patient using the package unique identifier. This may involve data processing device 105 querying a database containing a list of package unique identifiers corresponding to all packages registered with the system, to enable the drug(s) contained within the package to be looked up, e.g. database 110. Two exemplary database entries are shown below for a first package with unique identifier ‘Package1’ containing 10 tablets of drug X, and a second package with unique identifier ‘Package2’ containing 100 ml of a suspension containing drug Y.

| Package unique ID | Content | Drug format | Quantity |
|---|---|---|---|
| Package1 | Drug X | Tablet | 10 |
| Package2 | Drug Y | Suspension | 100 ml |

It will be appreciated that the format shown above is purely exemplary and significant deviations from this format can be made without departing from the scope of the present disclosure.

It is worth reiterating at this point that the package unique identifier corresponds to an individual package, and not to a batch of packages as manufactured and/or shipped concurrently. This is shown in the second exemplary database entry below, where two different packages from the same batch are assigned different package unique identifiers.

| Package unique ID | Content | Drug format | Quantity | Batch ID |
|---|---|---|---|---|
| Package1 | Drug X | Tablet | 10 | Batch1 |
| Package2 | Drug X | Tablet | 10 | Batch1 |

The package unique identifiers enable tracking of a package at the package level, rather than the batch level. This allows system 100 to determine exactly which drugs have been administered to the patient, i.e. at the level of individual capsules, tablets, bottles, etc., rather than at the batch level. Without being bound by theory, this is believed to result in improved determinations of patient compliance because the history of the specific instance of the drug that is actually administered to the patient can be determined in a manner that is not possible in the prior art.

In step 215, data processing device 105 stores in database 100 the identity of the drug as determined in step 210, the time of administration of the drug and the amount of the drug administered to the patient as provided in or otherwise derived from the message received in step 205, against the record associated with the patient. Optionally, step 215 can include storing any combination of the following against the patient record: one or more environmental parameters that characterise an environment experienced by the patient; behavioural data indicative of a behaviour of the patient; physiological data relating to the patient; and/or package data relating to the physical condition of the package associated with the patient. Reference is also made here to the discussion above in respect of items A) to D).

In optional step 220, data processing device 105 compares at least one pair of values stored in the patient record with the dosage regimen that is also stored in the patient record to calculate a compliance score indicative of the degree to which the patient has complied with the dosage regimen. The comparison will vary according to the specifics of the dosage regimen and the skilled person will be able to configure data processing device 105 to perform a comparison according to said specifics.

For example, data processing device 105 may compare the amount of drug administered to the patient with the amount proscribed by the dosage regimen and assign a compliance score based on the similarity between these two parameters. In another example involving a plurality of time values, data processing device 105 may determine a frequency of administration from the time values and compare this to a frequency specified in the dosage regimen, with a compliance score being assigned based on the similarity between these two parameters. Many modifications will be apparent to a skilled person having the benefit of the present disclosure.

As a further part of optional step 220, data processing device 105 can store the compliance score in database 100 against the patient record.

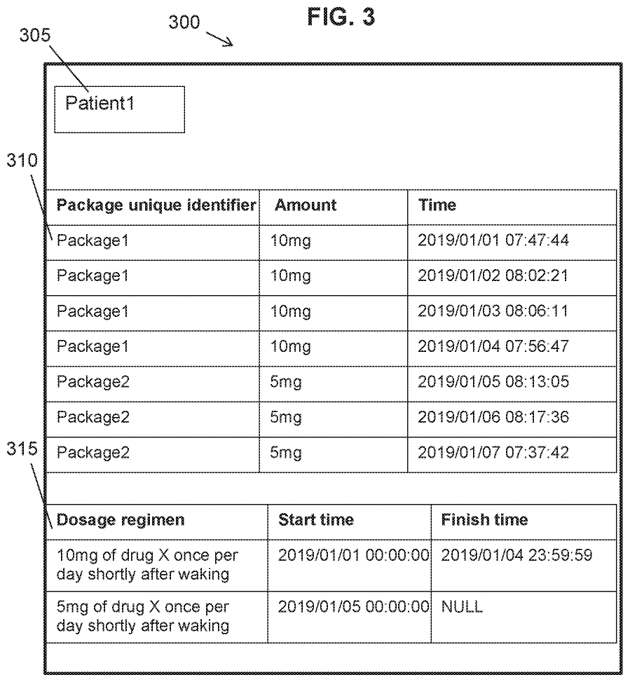

An exemplary patient record 300 according to an embodiment is shown in FIG. 3. Patient record 300 includes a patient unique identifier 305 (‘Patient1’) and a plurality of entries 310 that each include a package unique identifier, an amount of a drug administered to the patient and a time of administration. Other data can also be included in entry 310, e.g. an identification of the drug administered as is determinable from the package unique identifier in the manner discussed earlier in respect of step 210. Entries 310 can be populated as part of the storing process of step 215.

Patient record 300 also includes a dosage regimen section 315 that includes a dosage regimen associated with the patient. While it is possible to store only the current dosage regimen in patient record 300, in a preferred format patient record 300 includes a dosage regimen history. The dosage regimen history captures the revisions to the dosage regimen for the patient identified by patient unique identifier 305 that have been made over time. Optionally, a start time and finish time can be recorded against each iteration of the dosage regimen, where the start time indicates the time at which the dosage regimen became effective and the finish time indicates the time at which the dosage regimen ceased to be effective. Here, ‘effective’ is understood to mean that a patient would be dispensed medication according to the dosage regime if a dispensation request was received at some time between the start and finish times. An entity indicating no presence of data, e.g. ‘null’, entered as a finish time can indicate that the corresponding dosage regimen is currently in effect. This is purely exemplary and other suitable alternatives, such as a blank entry, will be apparent to a skilled person.

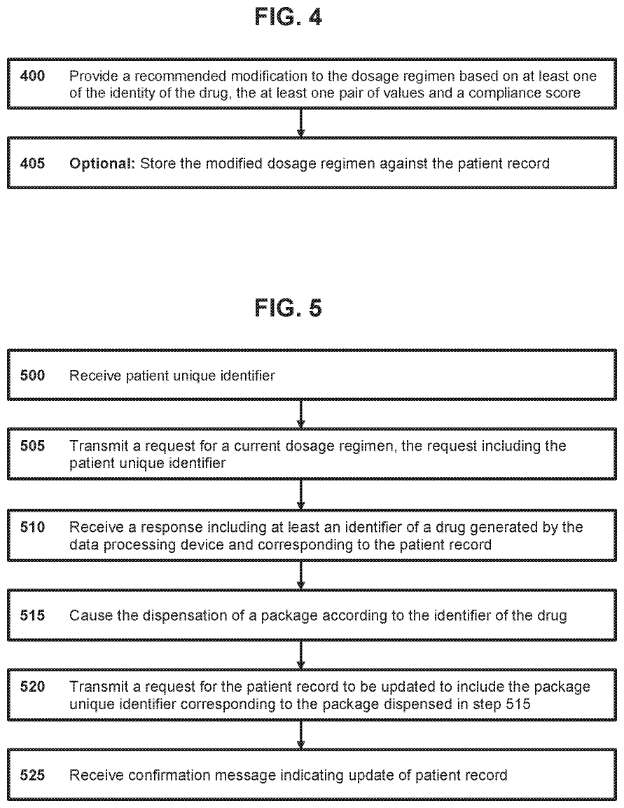

Revisions to a patient's dosage regimen can be recommended and optionally also applied by data processing device 105 following the process discussed in respect of FIG. 4 below.

It will be appreciated that patient record 300 has been illustrated in a manner that enables ready understanding by the reader. In practice patient record 300 may take a form that diverges significantly from the illustrated patient record 300, such as one or more tables in a relational database. It is thus understood that it is the data content of patient record, not the format in which the record is presented, that is important in the context of the present disclosure.

Without being bound by theory, it is believed that the data structures generated by the present disclosure and captured in the form of one or more patient records, which data structures record individual packages across one or more patients in combination with data that corresponds to actual administration events to said one or more patients, may enable presently unrecognised patterns in patient response to be detected. The resulting insights may enable recommended modifications to patient dosage regimes and/or to drug administration techniques, where the recommended modifications are tailored to individual patients, or to a subset of a total population of patients. Thus, the data structures described herein and presented in the form of a patient record such as patient record 300 advantageously support a so-called ‘personalised medicine’ framework.

It is further envisaged that a plurality of patient records will form a data structure that is particularly suited to analysis by machine learning algorithms such that the above-mentioned insights may be identified automatically. Database 110 may serve as a repository for a training dataset comprising a plurality of patient records of the type described herein, on which model(s) can be trained to generate proposed modifications to dosage regimens of individual patients.

Relating to this aspect of the disclosure, FIG. 4 shows a method suitable for generating a recommended modification to a dosage regimen and optionally modifying the dosage regimen based on the recommendation.

In step 400, data processing device 105 provides a recommended modification to the dosage regimen stored in a particular patient record based on at least one of the identity of the drug administered by the patient, the at least one pair of values stored in the patient record and/or a compliance score stored in the patient record.

Providing a recommended modification may include inputting any combination of data stored in the patient record into a recommendation module that is accessible to data processing device 105. The recommendation module comprises an algorithm configured to receive data from the patient record and, based on said data, generate a recommended modification to the dosage regimen.

The recommendation module may take the form of a model trained by one or more machine learning techniques. The data used to train the model may comprise a plurality of patient records. Additional data may be used to train the model, where the additional data takes the form of any combination of the data discussed above in relation to items A) to D). Any of the data discussed in relation to items A) to D) above may additionally or alternatively be used in step 400 in the provision of a recommended modification to the dosage regimen.

It will be appreciated that the inclusion of any of the data discussed in items A) to D) above enables patient environment, lifestyle, physiological response and/or package history to be taken into account when recommending a modification to the patient dosage regimen. This is made possible by the patient record disclosed herein, which serves as a rich repository of patient data for both training predictive models and serving as input to trained predictive models. Thus, the patient record as disclosed herein is considered to be particularly well suited to providing a ‘tailored’ or ‘personalised’ approach to medical treatment.

In optional step 405, data processing device 105 stores the modified dosage regimen as recommended in step 400 against the patient record. Referring to the exemplary patient record illustrated in FIG. 3, storing the modified dosage regimen may comprise the following steps: entering a finish time for the dosage regimen currently in effect, appending a new entry to the dosage regimen section 315, entering a description of the dosage regimen (e.g. as provided by a healthcare professional), and entering a start time of the new entry as equal to the time at which the new entry to dosage regimen section 315 was created.

It will be appreciated that carrying out step 405 has the effect of automatically updating the patient dosage regimen, such that patient administration events in the future may be in accordance with the updated dosage regimen.

The modified dosage regimen can be an improved dosage regimen, in the sense that the modified dosage regimen is deemed to be in some way superior to the original dosage regimen. Examples of improvements include: reduction in side effects, improvement in patient health or condition, improvement in efficacy of treatment, reduction in cost of provision of drug, etc. Other such improvements will be apparent to a skilled person having the benefit of the present disclosure.

A particular example of an improved dosage regimen may be the reduction in the amount of the drug that is administered to the patient. This may be accompanied by little or no corresponding drop in efficacy of the treatment.

FIG. 5 shows a method of dispensing a package according to an embodiment.

This method is described as being performed by clinician data processing device 130.

In step 500, clinician data processing device 130 receives a patient unique identifier. This may be received by manual entry of the patient unique identifier, or it may be received by an automated mechanism such as scanning of a QR code or barcode by clinician data processing device 130, reading of a RFID tag by clinician data processing device 130, etc. Other suitable mechanisms for receiving a patient unique identifier will be apparent to a skilled person having the benefit of the present disclosure.

In step 505, clinician data processing device 130 transmits a request for a dosage regimen stored in a patient record corresponding to the patient unique identifier to data processing device 105, the request including the patient unique identifier.

In step 510, clinician data processing device 130 receives a response from data processing device 105, the response including at least an identifier of a drug generated by data processing device 105 based on the patient record corresponding to the patient unique identifier transmitted in step 505. Optionally, the response also includes the patient unique identifier as retrieved from the patient record, so as to enable the clinician data processing device 130 to check that it has received the dosage regimen associated with the correct patient.

In step 515, the clinician data processing device 130 causes the dispensation of a package according to the identifier of the drug received in step 510. Clinician data processing device 130 may identify a suitable package by examining the drug identifier and identifying a package containing said drug.

Causing the dispensation of a package can include manual or automated dispensation.

In the case of manual dispensation, step 515 can include: displaying, on a display of clinician data processing device 130, a graphical user interface including at least an indication of the drug to be dispensed (e.g. an image of the package and/or a name of the drug) and a package identifier field configured for entry of a package unique identifier; retrieving a package containing the drug from a store, i.e. retrieving a package that matches the image and/or name displayed on the display; dispensing said package; and receiving a package unique identifier corresponding to the dispensed package. The package unique identifier may be supplied by manual data entry (e.g. typed using a keyboard of the clinician data processing device 130), or it may be supplied in an automated manner, e.g. a user scanning a barcode, QR code, RFID chip, or any other form of a machine-readable data encoding technique that is provided on the package that is being dispensed, where at least the package unique identifier is encoded by whichever machine-readable data encoding technique is used. It will be appreciated that the user in this case is likely to be a healthcare professional, e.g. a pharmacist or doctor.

In the case of automated dispensation, step 515 can include: transmitting a dispensation instruction to a dispensation device, the dispensation instruction including an identifier of a drug to be dispensed; and receiving a response from the dispensation device, the response including a package unique identifier corresponding to a package that is to be dispensed by the dispensation device. The dispensation device can be, for example, a computer controlled drug cabinet, locker or other such ‘smart’ apparatus that holds one or more drug packages. The dispensation device can alternatively be a device such as a printer that is configured to print a prescription including the package unique identifier, such that the prescription can subsequently be redeemed for the particular drug package corresponding to the package unique identifier. This method of dispensation may be particularly suited to a situation where direct involvement of a healthcare professional is not required, such as the dispensation of a repeat prescription. The dispensation device may itself dispense the package directly, e.g. by operating to cause the package corresponding to the package unique identifier to be dispensed.

In step 520, clinician data processing device 130 transmits a request for the patient record to be updated to include the package unique identifier corresponding to the package dispensed in step 515. The request is transmitted to data processing device 105.

In step 525, the clinician data processing device 130 receives a confirmation message from data processing device 105 that the patient record has been updated by data processing device 105.

It will be appreciated that the process of FIG. 5 advantageously ensures that a patient receives a package that corresponds to the latest dosage regimen associated with their patient record. This is because the package is dispensed based on a dosage regimen provided by data processing device 105 in step 510, which dosage regimen is the current dosage regimen stored in association with the patient record. This is another benefit that may be realised by a patient record as described herein.

A further modification to the process shown in FIG. 5 is also contemplated. In this modification, steps 500 to 510 proceed as discussed above. However, dispensation occurs in step 515 in either of the following ways:
Method 1 (manual dispensation): displaying, on a display of clinician data processing device 130, a graphical user interface including at least an indication of the drug to be dispensed (e.g. an image of the package and/or a name of the drug); retrieving a package containing the drug from a store, i.e. retrieving a package that matches the image and/or name displayed on the display; and dispensing said package.
Method 2 (automated dispensation): transmitting a dispensation instruction to a dispensation device, the dispensation instruction including an identifier of a drug to be dispensed; dispensing a package; and receiving a response from the dispensation device, the response indicating that a package containing the drug has been successfully dispensed.

In both cases, the difference here is that the package unique identifier has not been determined during dispensation. Instead, determination of the package unique identifier occurs in a modified form of step 520, which is modified such that patient data processing device 115 scans the package that was dispensed in step 515, obtains the corresponding package unique identifier and transmits the package unique identifier to data processing device 105. Step 525 is correspondingly modified such that the patient data processing device 115 receives the confirmation that the patient record has been updated by data processing device 105.

As another modification, it is also contemplated that the current stock of packages at a given healthcare institution (e.g. a particular pharmacy or hospital) could be known to data processing device 105 in advance. In this scenario, step 505 is modified such that either the patient data processing device 115 or the clinician data processing device 130 receives a response message that includes a package identifier corresponding to a package that is known by data processing device 105 to be currently held in a store of the healthcare institution and which contains the drug(s) in a correct form to satisfy the current dosage regimen as stored on the patient record. The package corresponding to the package unique identifier is then dispensed to the patient, either manually or automatically, and a confirmation that dispensation has occurred successfully is transmitted by either the patient data processing device 115 or the clinician data processing device 130 to data processing device 105. Data processing device 105 can then update the patient record to indicate that the package corresponding to the package unique identifier has been dispensed to the corresponding patient. Data processing device 105 can also update a database holding stock information for the healthcare institution, to indicate that the dispensed package is no longer held in the store of the healthcare institution at said location. FIG. 6 shows an exemplary embodiment of a package 600 as may be used in connection with the present disclosure. Package 600 is an example of a ‘smart’ package that is capable of detecting administration events and reporting pertinent information to patient data processing device 115.

Package 600 takes the form of a blister pack and is illustrated from the back surface thereof. The back surface of the blister pack is understood to be the side that includes the blisters, and opposite to the side that contains a removable covering (usually foil) that is broken when a force is applied to the blister to push the drug capsule stored in the blister out through the removable covering.

Package includes an embedded data processing device in the form of microprocessor 605, a transmitter or transceiver in the form of antenna 610 and a plurality of blisters 615a to 615d. Four blisters are shown, but it will be appreciated that any number of blisters, including one blister, can be present in package 600. Microprocessor 605 and antenna 610 are known in the art per se.

The structure of the package itself, excepting the electronic components as discussed in detail below, is known in the art per se. Further information on these aspects of package 600 is thus omitted here in the interests of clarity and brevity.

Microprocessor 605 is electrically coupled to antenna 610 and each of blisters 615a-615d via respective electrical wires, represented by lines in FIG. 6. In the illustrated embodiment antenna 610 and the electrical wires are formed of electrically conductive traces that are laid on the back surface of package 600. Other techniques suitable for forming breakable wires on the back surface of package 600 can alternatively be used, e.g. embedding the wires within the removable covering and/or the structure of the package itself. Each wire traversing a blister comprises a separate electrical circuit that couples to microprocessor 605 at each end.

Microprocessor 605 is configured to control antenna 610 to enable communication with an external device such as patient data processing device 115 or clinician data processing device 130. Antenna 610 can be a NFC antenna, a Bluetooth antenna, preferably a Bluetooth Low Energy (BLE) antenna, or a radio transmitter or transceiver suitable for communication over a cellular network, e.g. a SIM card and radio frequency transceiver.

Microprocessor 605 may be coupled to, or include, a timing circuit (not shown) that is configured to keep track of time. This may be, for example, a clock chip or clock integrated circuit as are known in the art per se, which circuit is configured to provide a current time (e.g. a UTC time) on request by microprocessor 605.

Microprocessor 605 includes a memory module configured to store data in advance of it being transmitted via antenna 610. The memory module may store the package unique identifier corresponding to package 600 so that it is available for transmittal to another device, e.g. patient data processing device 115, when said device is brought within communication range of antenna 610. Alternatively, the package unique identifier may be provided in a non-electronic format, e.g. a barcode, QR code or other such visual indicia formed on a surface of package 600.

The memory module is configured to store a log file. The log file comprises a set of blister unique identifiers that uniquely identify each blister, and a status of each blister. The status is indicative of an open or closed state of the blister. The status for a given blister is determined by microprocessor 605 based upon whether or not an electrical signal can be successfully transmitted via the respective circuit corresponding to said blister. The log comprises a timestamp associated with each status, where each timestamp indicates the time at which the currently recorded status of the respective blister was most recently determined.

A power source (not shown) such as a battery can be included in package 600. Alternatively, package 600 may be a passive device that is powered using an inductive power supply (not shown) that is capable of generating electrical power using the induction effect when present in an electromagnetic field generated by another device, e.g. patient data processing device 115 or clinician data processing device 130.

Each blister includes a removable portion that can be removed by a user from the front surface of package 600 in order to gain access to the respective drug capsule. Typically, removal is achieved by applying pressure to the blister at the back surface of package 600 to force the contents of the blister through the removable layer, which is typically foil. As can be seen from FIG. 6, a respective wire traverses the extent of each removable portion.

As illustrated in FIG. 6, blisters 615a to 615c are unopened and thus each include a pharmaceutical composition which in this exemplary case is a drug capsule. The wires traversing each one of blisters 615a to 615c are unbroken such that they readily carry an electric current.

However, blister 615d has been opened and the capsule removed. The action of opening the blister 615d causes part or all of the associated removable portion to be torn away from the package 600. This in turn breaks the wire traversing the removable portion, such that the circuit associated with blister 615d will no longer conduct electricity.

Microprocessor 605 is configured to transmit a signal along each electrical circuit that corresponds to a respective blister. In the configuration shown in FIG. 6, signals associated with blisters 615a to 615c will be received by microprocessor 605. Reception of each signal indicates that the removable portion of these blisters is intact, which in turn indicates that the respective drug capsules have not yet been administered. However, the removal of the removable portion of blister 615d breaks the wire associated with this blister, such that no signal is received in respect of blister 615d by microprocessor 605. In this way, microprocessor 605 can identify that the capsule originally stored in blister 615d has been administered.

Microprocessor 605 can be configured to attempt to transmit signals along each respective blister circuit and to identify any circuit(s) for which the transmission attempt fails. The transmission attempts can be made periodically, e.g. once a minute, or they can be made in response to detection of patient data processing device 115 by antenna 610.

On discovery of a failed transmission attempt, microprocessor 605 can be configured to log the time at which the transmission attempt failed in the log, this time being understood to be closely correlated with or approximately equal to the time at which the drug capsule contained within the blister associated with the broken circuit was opened. The logged times can be transmitted to another device via antenna 610 as and when said device is within communication range of antenna 610.

Package 600 can additionally or alternatively include one or more sensors (not shown) that are configured to determine one or more parameters relating to the environment of package 600. One example of a suitable sensor is an accelerometer that is coupled to microprocessor 605. Data from the accelerometer that is indicative of motion undergone by the package 600 may be stored in a memory embedded within the package for transmittal to an external device such as patient data processing device 115.

Other sensors include: a temperature sensor, a light level sensor, a location sensor (e.g. GPS sensor), a moisture sensor, a sound meter, and any other sensor known to the skilled person. Data from any of these sensors can be stored in the memory for transmittal to a device such as patient data processing device 115 as and when it is within range of antenna 610.

Microprocessor 605 may be configured to record any combination of the following parameters in the log file: a physical location of the blister pack at the time the at least one electrical wire was broken; and at least one parameter characterising the local environment of the blister pack at the time the at least one electrical wire was broken.

Each of the drug capsules contained within blisters 615a to 615c may be identical in the sense that the drug capsules each comprise the same amount of a particular drug. Alternatively, one of the blisters, e.g. blister 615a, may contain a drug capsule comprising a first amount of the drug and a different one of the blisters, e.g. blister 615b, may contain a drug capsules comprising a second, different amount of the drug. As a further alternative, one of the blisters, e.g. blister 615a, may contain a drug capsule comprising a first drug and a different one of the blisters, e.g. blister 615b, may contain a drug capsule comprising a second, different drug. Combinations are possible, such that more than one drug is provided at more than one dosage level in a single blister pack. It will be appreciated that these concepts can be extended, e.g. three or more different drugs can be provided in a single package, and/or three or more different dosage amounts of a single drug can be provided in a single package.

Blister packs containing different dosage amounts of the same drug as described in the immediately preceding paragraph may be advantageously used in cases where it is expected that the dosage regimen will be adjusted over a timescale that is shorter than the expected lifetime of a given package, i.e. the patient will not be expected to finish the package before their dosage regimen is modified. The different dosages enable administration of more than one drug capsule simultaneously to fine tune the total amount of the drug delivered in a single administration event. For example, providing capsules comprising a given drug in both a 5 mg format and a 2 mg format enables the total amount of the drug administered in a single administration event to be adjusted in increments of 2 mg but also without requiring an excessive number of individual capsules to be administered per administration event.

Blister packs containing more than one type of drug capsule find particular utility in situations where a dosage regimen requires administration of a combination of two or more drugs. In this case, and also in the case where different dosage amounts of a single drug are present, the patient data processing device 115 may assist in the identification of the appropriate capsule(s) to administer based on the current dosage regimen as stored in the patient record, e.g. by providing on-screen instructions to the patient or other person, e.g. “please administer one red pill and one yellow pill”, and/or by use of augmented reality, e.g. providing an overlay on image of the blister pack on a display of the patient data processing device 115, which overlay indicates the capsule(s) that should be administered.

It will be appreciated that any of the methods described herein, or parts thereof, can be encoded by computer-readable instructions and stored on a non-transitory computer-readable medium. Any part of the subject matter described above can thus be implemented by a computer executing appropriate instructions stored on a non-transitory computer-readable medium. A computer readable medium storing such instruction is thus also within the scope of the present disclosure.

The foregoing discussion discloses embodiments in accordance with the present disclosure. As will be understood, the approaches, methods, techniques, materials, devices, and so forth disclosed herein may be embodied in additional embodiments as understood by those of skill in the art, it is the intention of this application to encompass and include such variation. Accordingly, this disclosure is illustrative and should not be taken as limiting the scope of the following claims.

What is claimed is:

1. A computer-implemented method for monitoring the adherence of a patient to a dosage regimen, the method comprising:
receiving, by a data processing device, a patient unique identifier that enables the patient to be uniquely identified and associated with a patient record;
receiving, by the data processing device, a message, wherein the message includes:
a package unique identifier that enables a package that a drug administered to the patient was stored within to be uniquely identified; and
at least one pair of values, each pair of values corresponding to an administration event and indicating an amount of the drug administered to the patient and a time of administration of the amount of the drug to the patient;
the method further comprising:
identifying, by the data processing device, the drug administered to the patient using the package unique identifier; and
storing, in a database, the identity of the drug and the at least one pair of values against the patient record, the patient record including the dosage regimen associated with the patient.

2. The computer-implemented method of claim 1, further comprising:
comparing, by the data processing device, the at least one pair of values with the dosage regimen;
calculating, by the data processing device and based on the comparing, a compliance score indicative of the degree to which the patient has complied with the dosage regimen; and
storing, by the data processing device, the compliance score in the database against the patient record.

3. The computer-implemented method of claim 1, further comprising:
providing, by the data processing device, a recommended modification to the dosage regimen based on at least one of the identity of the drug and the at least one pair of values.

4. The computer-implemented method of claim 2, further comprising:
providing, by the data processing device, a recommended modification to the dosage regimen based on any combination of: the identity of the drug, the at least one pair of values, and the compliance score.

5. The computer-implemented method of claim 1, further comprising:
receiving, by the data processing device, one or more environmental parameters that characterise an environment experienced by the patient;
storing, in the database, the one or more environmental parameters against the patient record; and
providing, by the data processing device, a recommended modification to the dosage regimen based on at least one of the one or more environmental parameters.

6. The computer-implemented method of claim 1, further comprising:
receiving, by the data processing device, behavioural data indicative of a behaviour of the patient;
storing, in the database against the patient record, the behavioural data; and
providing, by the data processing device, a recommended modification to the dosage regimen based on at least the behavioural data.

7. The computer-implemented method of claim 1, further comprising:
receiving, by the data processing device, physiological data relating to the patient;
storing, in the database against the patient record, the physiological data; and
providing, by the data processing device, a recommended modification to the dosage regimen based on at least the physiological data.

8. The computer-implemented method of claim 1, further comprising:
receiving, by the data processing device, package data relating to the physical condition of the package;
storing, in the database against the patient record, the package data; and
providing, by the data processing device, a recommended modification to the dosage regimen based on at least the package data.

9. The computer-implemented method of claim 1, further comprising:
storing, by the data processing device, a modified dosage regimen against the patient record.

10. The computer-implemented method of claim 9, further comprising:
generating, by the data processing device, the modified dosage regimen based on a recommended modification to the dosage regimen; or
receiving, at the data processing device, the patient unique identifier and the modified dosage regimen.

11. The computer-implemented method of claim 10, further comprising:
transmitting, by the data processing device, a message to a second data processing device, the message including the modified dosage regimen.

12. The computer-implemented method of claim 11, wherein the second data processing device is a patient data processing device or a healthcare provider data processing device.

13. The computer-implemented method of claim 10, wherein the modified dosage regimen comprises an improved dosage regimen.

14. The computer-implemented method of claim 13, wherein the improved dosage regimen is a reduction in an amount of the drug.

15. The computer-implemented method of claim 11, wherein the second data processing device is any one of: a mobile telephone, a tablet computer, a desktop computer, a voice-activated computing system, a laptop, a gaming system, a vehicular computing system, a wearable device, a smart watch, a smart television, an internet of things device, a medicament-dispensing device and a device including a drug pump.

16. The computer-implemented method of claim 1, wherein the package is a carton, a blister pack, a dosette box, a bottle, a tube, a syringe, a pre-filled syringe, a cartridge, a vial or a canister.

17. The computer-implemented method of claim 1, wherein the package is a blister pack comprising:
at least one blister in which the drug is stored;
at least one electrical wire traversing the blister;
an embedded data processing device embedded in the blister pack and coupled to the at least one electrical wire;
at least one memory module coupled to the embedded data processing device;
a transmitter or transceiver coupled to the embedded data processing device; and
a mechanism for encoding the package unique identifier;
the method further comprising:
detecting, by the embedded data processing device, a break in the at least one electrical wire;
storing, by the embedded data processing device in the at least one memory module, at least a time at which the at least one electrical wire was broken and a blister identifier associated with the blister traversed by the at least one electrical wire that was broken.

18. The computer-implemented method of claim 17, wherein the package comprises at least one blister containing the drug in a first amount and at least one blister containing the drug in a second amount.

19. The computer-implemented method of claim 17, wherein the package comprises at least one blister containing the drug and at least one blister containing a second, different drug.

20. The computer-implemented method of claim 17, further comprising:
recording, by the embedded data processing device in the at least one memory module, any combination of the following parameters: a physical location of the blister pack at the time the at least one electrical wire was broken; and at least one parameter characterising the local environment of the blister pack at the time the at least one electrical wire was broken.

21. The computer-implemented method of claim 17, wherein the at least one pair of values is based upon data stored in the at least one memory module.

22. The computer-implemented method of claim 17, wherein the mechanism for encoding the package unique identifier is any one or more of: a visual indicator printed on the blister pack; a NFC-enabled integrated circuit; and a Bluetooth-enabled integrated circuit.

23. The computer-implemented method of claim 17, wherein the transmitter or transceiver is one of:
a Bluetooth transmitter or transceiver;
a NFC transmitter or transceiver; and
a radio transmitter or transceiver suitable for communication over a cellular network.

24. The computer-implemented method of claim 17, wherein the blister pack comprises at least a first blister and a second blister, the first blister containing a first tablet comprising the drug in a first amount and the second blister containing a second tablet comprising the drug in a second, different amount.

25. The computer-implemented method of claim 17, wherein the blister pack comprises at least a first blister and a second blister, the first blister containing a first tablet comprising the drug and the second blister containing a second tablet comprising a second, different drug.

26. The computer-implemented method of claim 17, wherein the blister pack comprises at least a first blister, a second blister, and a third blister, the first blister containing a first tablet comprising the drug in a first amount, the second blister containing a second tablet comprising the drug in a second, different amount and the third blister containing a third tablet comprising a second, different drug.

27. The computer-implemented method of claim 1, further comprising:
receiving a request for the patient record to be updated to include a second package unique identifier corresponding to a package that has been dispensed to the patient; and
storing, by the data processing device, the second package unique identifier against the patient record.

28. The computer-implemented method of claim 1, further comprising:
receiving, by the data processing device, a request to dispense medication, the request including the patient unique identifier;
transmitting, by the data processing device, an instruction to dispense a package selected based on the dosage regimen that is stored in the patient record corresponding to the patient unique identifier, the instruction including a second package unique identifier corresponding to the selected package; and
storing the second package unique identifier against the patient record.

29. The computer-implemented method of claim 1, wherein the patient is enrolled in a clinical trial and the patient unique identifier was assigned to the patient during enrolment in the clinical trial.

30. A non-transitory computer-readable medium comprising program instructions that, when executed by a data processing device, cause the data processing device to perform the following actions:
receive a patient unique identifier that enables the patient to be uniquely identified and associated with a patient record;
receive a message, wherein the message includes:
a package unique identifier that enables a package that a drug administered to the patient was stored within to be uniquely identified; and
at least one pair of values, each pair of values corresponding to an administration event and indicating an amount of the drug administered to the patient and a time of administration of the amount of the drug to the patient;
identify the drug administered to the patient using the package unique identifier; and
store, in a database, the identity of the drug and the at least one pair of values against the patient record, the patient record including the dosage regimen associated with the patient.

31. A data processing device that is configured to perform the following actions:
receive a patient unique identifier that enables the patient to be uniquely identified and associated with a patient record;
receive a message, wherein the message includes:
a package unique identifier that enables a package that a drug administered to the patient was stored within to be uniquely identified; and
at least one pair of values, each pair of values corresponding to an administration event and indicating an amount of the drug administered to the patient and a time of administration of the amount of the drug to the patient;
identify the drug administered to the patient using the package unique identifier; and
store, in a database, the identity of the drug and the at least one pair of values against the patient record, the patient record including the dosage regimen associated with the patient.
